(19) United States
(12) Patent Application Publication
(10) Pub. No.: US 2008/0103409 A1
(43) Pub. Date: May 1, 2008

(54) Methodology for developing a new approach for visual information cognition

(76) Inventor: Mark Komissarov, Brooklyn, NY (US)

Correspondence Address: COOPER & DUNHAM, LLP, 1185 AVENUE OF THE AMERICAS, NEW YORK, NY, 10036, US

(21) Appl. No.: 11/973,784

(22) Filed: Oct. 10, 2007

Related U.S. Application Data
(60) Provisional application No. 60/854,231, filed on Oct. 25, 2006.

(51) Int. Cl. A61B 13/00

(52) U.S. Cl. 600/558

(57) ABSTRACT

A system and method defining a new approach to visual information cognition, and methodology for teaching individuals to use this information to attain vision and increase brain usage by bypassing the sensory organs typically used like eyesight for processing this information. The system and method enables individuals to develop a part of the brain which is typically underutilized with resultant development of new channels to attain vision, while simultaneously potentially increasing IQ and improving memory.

This application claims the benefit of U.S. Provisional Application No. 60/854,231, filed Oct. 25, 2006, the content of which is hereby incorporated by reference into this application.

### BACKGROUND

Carl Jung, in his book, Psychological Types, first published in English in 1922, defined the polar-opposites elements of introversion and extroversion as two ways of relating to the world. He also defined sensing and intuition as two ways to perceive, and thinking and feeling as two ways of judging. His research was based on psychiatric treatments of his individual medical patients.

Extending Jung's work on perception, environmental information input to the brain is typically organized by five special senses and a few non-specific ones. The five special senses are: vision, hearing, balance, smell and taste. They are “special” because the actual sensors (receptors) are localized and specialized (physically, chemically and anatomically) to acquire specific environmental data, but within a limited range of changes. For example, humans cannot see in the infrared part of the spectrum (as do snakes) or the ultraviolet range (as do some insects). Similarly, humans cannot hear in the infra- or ultra-sonic ranges of sound frequency as do, respectively, elephants or bats.

Non-specific senses for mechanical signal, thermal changes, or pain, do not have a specific location or specialized apparatus for reception. Nevertheless, all non-specific senses are also limited in terms of the ranges of environmental information that can be sensed (frequency of vibration, temperature range, etc.).

People usually do not think about such natural behavioral acts like breathing or digestion as fully “automatic”, internally “built-in” processes. Even if we think about them, we cannot stop or permanently change them. Walking, swimming, riding a bike or driving a car are other examples of very complex biomechanical processes that also use multiple sensory and motor coordination, but we learn them early in our lives; performing them also almost naturally (without thinking about each component), quickly and with great precision and efficiency.

The present invention provides means for efficiently training the brain to carry out new tasks and perceive and utilize new information “automatically” using a new non-specific channel of sensing. It is hypothised that this invention results in strong activation in areas of the primary visual cortex. This means that after training, a blindfolded or blind person's brain begins to use the most sophisticated analytical part of the cortex for analysis of information displayed during visual tasks. Before training, it is contemplated that these areas were not active. The activation of normal analytical resources (e.g. the ‘visual’ part of the brain) in response to artificial sensory stimulation was “automatic” in that it did not rely on the use of the eyes for directing the information to the primary visual cortex.

Mediated by the receptors, energy transduced is encoded as neural pulse trains. In this manner, the brain is able to recreate “visual” images that originate when the viewing person is blindfolded. Indeed, after sufficient training subjects, who were blind, reported experiencing images. They learned to make perceptual judgments using visual means of analysis, such as perspective, parallax, looming and zooming, and depth judgments. These have included reading books, facial recognition, and accurate judgment of speed and direction of a rolling ball with 100% accuracy in catching the ball as it rolls.

We see with the brain, not the eyes; images that pass through our pupils go no further than the retina. From there image information travels to the rest of the brain by means of coded pulse trains, and the brain, being highly plastic, can learn to interpret them in visual terms. Perceptual levels of the brain interpret the spatially encoded neural activity, modified and augmented by nonsynaptic and other brain plasticity mechanisms. However, the cognitive value of that information is not merely a process of image analysis. Perception of the image relies on memory, learning, contextual interpretation (e.g. we perceive intent of the driver in the slight lateral movements of a car in front of us on the highway), cultural, and other social factors that are probably exclusively human characteristics.

The systems of the present invention may be characterized as a humanistic intelligence system tied into a hypothetical Center of Information processing in the brain. This is made possible by “sensory plasticity”, the capacity of the brain to reorganize when there is: (a) functional demand, (b) the sensor technology to fill that demand, and (c) the training and psychosocial factors that support the functional demand. To constitute such a system then, it is only necessary to present environmental information in a form of energy or stimuli that can be mediated by the brain, to determine the origin of the information.

A simple example of sensory substitution system is a blind person navigating with a long cane, who perceives a step, a curb, a foot and a puddle of water, but during those perceptual tasks is unaware of any sensation in the hand (in which the biological sensors are located), or of moving the arm and hand holding the cane. Rather, he perceives elements in his environment as mental images derived from tactile information originating from the tip of the cane. This can now be extended into other domains with systems of the present invention associated with new sensory inputs.

Psychoneuroimmunology explores how the brain and body interact through the immune system to influence health and disease. Until only a few decades ago, the workings of the brain were unknown, seemingly impenetrable to the curious eyes of researchers. But within the last decade, a new wave of mind body research has been made possible by ultramodern brain imaging techniques as well as new laboratory methods to decode the immune system's chemical messengers.

The major breakthrough, in terms of understanding the mind-body relationship, was the discovery that our immune system uses a two-way flow of information. This is achieved primarily by using a vocabulary comprised of specialized messenger molecules (i.e., peptides and cytokines) that flow via immune cells between the brain and the body. In other words, it is believed that immune cells may have a primitive sensory capacity and, like their nerve cell counterparts, can detect threats—real or impending—and then relay chemical messages to alert the brain or to modify its current activities.

Another key discovery concerning the mind-body relationship is that the brain and its pathways of nerves are far more adaptable (scientists use the term “plastic”) than any other part of the body. Briefly, this adaptability or “plasticity” phenomenon refers to the fact that the brain reacts constantly to outside stimuli and rewires itself, when necessary, according to information it receives. This rewiring concept is fascinating and may help to explain how emotional experiences develop and are maintained in memory. The re-wiring idea basically says that “feeling an emotion” is a product of the brain's adaptable interconnectedness.

Another example of the brain-plasticity concept is demonstrated by the relationship between emotional depression and chronic pain. Here, the constant barrage of painful stimuli to the brain essentially causes a rewiring of the brain's chemical pathways, which try to dampen the incoming pain signals. Unfortunately, the system used to suppress the pain, if allowed to run long enough, opens the pathway to emotional depression.

Healing resources exist within the brain that can be called upon to repair, or assist in repairing, the physical body or its emotional state. The power of this mind-body resource is dramatically demonstrated by comparing the placebo and nocebo effects.

The placebo effect is typically defined as the healing response initiated by a fake therapy (e.g., sugar pill) and is every bit as real in those individuals receiving the fake therapy as it is in those receiving the real therapy. The nocebo effect is lesser known but is somewhat more dramatic in its unhealthful effect upon a person. This negative health effect is seen in a real-life case in which an individual was convinced by her doctor that she had a fatal disease (in this case, Acquired Immunodeficiency Syndrome (AIDS)) although she really did not have an HIV infection (the doctor had been given the wrong blood test results). As time passed, the patient developed the physical signs of AIDS. Fortunately, she moved to a new city and had to change doctors. The new doctor discovered the error and informed the patient. She spontaneously recovered.

There is an increasing body of evidence showing that the mind is capable of mobilizing the body's immune system. It appears that this mobilization is achieved through the release of peptides and cytokines. One's state of immune system preparedness is, in part, determined by which of these peptides and cytokines has the most influence when they are transmitted from the brain. Worry, fear, and loss of sleep, for example, tend to promote the release of the peptides and cytokines that at first over-stimulate the immune system and then later exhaust it. This concept is also a possible explanation for blindsight as discussed below.

Conscious visual perception occurs in the back of the brain in an area called the occipital cortex. Each side of the brain has such an area—the left occipital cortex being where the right side of the visual field is perceived and the right occipital cortex performing the same function for the left side of the visual field. This much is simply deduced from accidental damage to these areas. Such damage will result in the victim losing the ability see part or all of the corresponding visual field depending on the extent of the damage.

The left eye is not just connected to the right occipital cortex and visa versa. The connections of the optic nerves do not lead directly to the occipital cortex. Indeed they are arranged so that each eye feeds half of the information it receives each side of the brain. Therefore the effect of loss of the visual cortex on one side of the brain is different from loss of an eye or its optic nerve. Also, whereas damage to the eye affects the whole visual field, damage to the visual cortex on one side effect only half of the visual field as seen in both eyes.

If the entire occipital cortex is not completely destroyed the victim will report a loss of visual perception in part of the visual field. The effect is similar to the blind spot in the visual field created where the optic nerve exits the eye and can be quite large. With the victim's cooperation an examination can map the size and position of the resulting blind spot. However victims of stroke in the occipital cortex often lose visual perception in the entire left or right visual field. Destruction of both sides of the visual cortex will result in complete blindness.

A notable observation in some victims of occipital cortical damage due to stroke is that while they experience no conscious visual perception in the blinded area they nevertheless exhibit a remarkable ability to guess accurately about its content. Experiments show that such subjects do much better than average in guessing if a light has been flashed in the blinded area or whether a shape displayed there was a circle or square or the direction of a movement there. This is the phenomenon of blindsight. Blindsight victims may be blind but they somehow receive information through their eyes.

The obvious reaction on first hearing of blindsight is that it is too good to be true—surely experimenters or subjects (or both) who report blindsight are either faking it or have been fooled by hysterical blindness. But with careful analysis both possibilities can be ruled out. For instance both fakers and patients diagnosed as hysterically blind tend to do very badly in perceiving their environment—stereotypically “bumping into the furniture” much more even than do really blind subjects. Additionally blindsight victims are also observed to have damage in the occipital cortex consistent with the blindness they describe.

Blindsight victims are not aware of their extra-conscious visual perception unless it is discovered and pointed out to them. They do not just volunteer that they have this ability or these perceptions. Once discovered blindsight can be trained so that the victim's ability to use it in experimental situations improves. But it does not replace conscious visual perception with a kind of pseudo-perception. The blind sight victim is never aware of whatever visual processing is occurring when they guess what they cannot see. They are still guessing.

Blindsight should not be surprising considering the many connections in the brain into which the optic nerve feeds. Information from the eyes is relayed many times before reaching the seat of conscious visual perception (wherever that may be). There is plenty of opportunity and ample evidence for visual processing along the way. What is surprising is that many of the functions that we equate with visual perception such as sensing the presence or absence of light, detecting shapes, color and detecting motion apparently occur without the conscious visual perception that we equate with seeing.

Conscious visual perception is such a powerful experience that we believe it to be defining and all encompassing of any and all visual perception. We do not notice that some of the visual processing that occurs in our brains occurs outside of this experience. We assign all this processing and the perceptions that result to conscious visual perception as a matter of course. But take away conscious perception and the additional visual processing done elsewhere in the brain is still left.

The fact is that apparently we can in some sense see without conscious visual perception. Though blindsight is clearly not the real thing what is it and what does it say about the real thing? Blindsight allows us a glimpse of unconscious processes that might be called preconscious visual perception. It may be that this preconscious perception is a functional part of conscious visual perception. On the other hand it may just be a vestigial capability that is no longer a part of conscious visual perception. In either case it raises the possibility of visual perception that operates entirely at an unconscious level.

Blindsight suggests that the mental processing that makes up visual perception is not a single capacity located in a single area of the brain. This is an idea that may well apply to more than just visual perception. The mind as a whole might be distributed throughout the brain and its units function in a coordinated but semiautonomous way. Loss of one processing area might result in a fragmented mind. In such a fragmented mind a whole faculty like conscious vision might be lost but not its components. In that case the mind would be unable to construct the whole perception but might still have access to its components. This seems to be precisely what we see in blindsight.

In the course of research into blindsight, based upon the concept of the brains ‘plasticity’, I have invented a methodology that can be used to teach individuals how to see and read with their eyes blindfolded by developing the faculty to obtain images through their foreheads. In testing on blind individuals, it has been possible for them to reobtain some limited vision. Furthermore I have noticed that individuals who read and absorb information through this technique are potentially developing an area of the brain which to date has been under utilized with the potential benefit of increased intelligence quotients and brain utilization.

### SUMMARY OF THE INVENTION

A method is provided for altering a visual cognitive ability in a human comprising:
(a) eliciting the human to imagine that he or she possesses an optical sensory ability that operates when the human is sight-deprived;
(b) presenting the sight-deprived human with an optical sensory stimulus for a time sufficient that the human perceives the presence of the optical stimulus; and
(c) eliciting, within a few seconds of the human perceiving the presence of the optical stimulus, a description of the optical stimulus,
so as to thereby alter the visual cognitive ability of the human.

A method is provided for eliciting a visual cognitive ability in a congenitally blind human comprising:
(a) eliciting the human to imagine that he or she possesses an optical sensory ability;
(b) (i) presenting the human with a first optical stimulus for a time sufficient that the human perceives the presence of the optical stimulus and (ii) informing the human of the nature of the optical stimulus;
(c) eliciting the human to familiarize himself/herself with a thought elicited by the optical stimulus;
(d) repeating steps (b) and (c) with a further optical sensory stimulus;
(e) repeating steps (b) to (d) a sufficient number of times until the human is able to identify the nature of the optical stimulus without being informed of its nature,
thereby eliciting the visual cognitive ability in the congenitally blind human.

The present invention covers a system and method defining a new approach to visual information cognition, and methodology for teaching individuals to use this information to attain vision and increase brain usage by bypassing the sensory organs used like eyesight for processing this information. The system and method enables individuals to develop a part of the brain which is typically underutilized with resultant development of new channels to attain vision, while simultaneously potentially increase IQ and improve memory.

The principal discovery concerning the mind-body relationship is that the brain and its pathways of nerves are far more adaptable (“plastic”) than any other part of the body. Briefly, this adaptability or “plasticity” phenomenon refers to the fact that the brain reacts constantly to outside stimuli and rewires itself, when necessary, according to information it receives. I have taken this key discovery and developed a methodology to enable people through correct stimuli and teaching to be able to rewire their brains as discussed in the detailed description below.

For the sake of simplicity I have hypothesized that the human brain contains a center which is capable of accepting information directly, bypassing the sensory organs. I call it the Center of Information Processing (CIP). This CIP is always in a passive state and does not take an active role in delivering information.

I consider that it is extremely important to learn how to activate the human CIP and include it in the process of receiving information, since the information reaching the CIP is always accurate and complete. A person receiving such information will cease to make incorrect decisions.

This invention provides:

1) A system and method for understanding and developing the information perception and visual cognitive abilities of an individual outside the normal senses of the eyes for “Real sight”—the reception, by the brain, of visual information regarding physical objects in the field of real sight.
2) A system and method for understanding and developing the information perception and visual cognitive abilities of an individual outside the normal senses of the eyes for “Seeing through physical barriers”—The reception, by the brain, of information regarding an object, hidden by a physical barrier.
3) A system and method for understanding and developing the information perception and visual cognitive abilities of an individual outside the normal senses of the eyes for “Farsightedness”—The reception of information, by the brain, of an object located at a significant distance.
4) A system and method for understanding and developing the information perception and visual cognitive abilities of an individual outside the normal senses of the eyes for “Seeing the past”—The reception of information, by the brain, of events that took place in the past.
5) A system and method for understanding and developing the information perception and visual cognitive abilities of an individual outside the normal senses of the eyes for “Retrieving information from any type of carrier.”—The reception of information, by the brain, from such carriers as computer disks, compact disks, tapes, etc.

### DETAILED DESCRIPTION OF PREFERRED EMBODIMENTS OF THE INVENTION

A method is provided for altering a visual cognitive ability in a human comprising:
(a) eliciting the human to imagine that he or she possesses an optical sensory ability that operates when the human is sight-deprived;
(b) presenting the sight-deprived human with an optical sensory stimulus for a time sufficient that the human perceives the presence of the optical stimulus; and
(c) eliciting, within a few seconds of the human perceiving the presence of the optical stimulus, a description of the optical stimulus,
so as to thereby alter the visual cognitive ability of the human.

In an embodiment, in step (c), initiation of the description is elicited within 5 seconds.

In another embodiment, in step (c), initiation of the description is elicited within 2.5 seconds.

In embodiments the optical stimulus is paper and is substantially black, substantially white, or substantially colored.

In an embodiment, the optical stimulus is text.

The instant method is provided further comprising, prior to step (a), the steps of:
(i) exposing the human to a demonstration of a second human describing an optical stimulus, wherein the second human is denied sight of the optical stimulus; and
(ii) applying a sight-depriving means to the human.

A method is provided for eliciting a visual cognitive ability in a congenitally blind human comprising:
(a) eliciting the human to imagine that he or she possesses an optical sensory ability;
(b) (i) presenting the human with a first optical stimulus for a time sufficient that the human perceives the presence of the optical stimulus and (ii) informing the human of the nature of the optical stimulus;
(c) eliciting the human to familiarize himself/herself with a thought elicited by the optical stimulus;
(d) repeating steps (b) and (c) with a further optical sensory stimulus;
(e) repeating steps (b) to (d) a sufficient number of times until the human is able to identify the nature of the optical stimulus without being informed of its nature,
thereby eliciting the visual cognitive ability in the congenitally blind human.

In embodiments, the first or further optically stimulus is paper which is substantially black, substantially white, or substantially colored.

The activation method for the Center of Information Reception in the human brain is described below.

### Activation Method for the Center of Information Reception in a Human Brain.

The activation method for the Center of Information Reception (CIP) is based in on the removal of the psychological barrier in the human brain which prohibits the perception of the surrounding world and in arousing the brain to receive information through a new channel.

The presence of a psychological barrier in the human brain is based on his preconception that the only way information is received from the outside world is with the aid of the five sensory organs. This preconception has in it a “genetic” foundation and is based on a hundred million years of evolution on Earth. The goal of this method is to overcome this settled preconception and to open for the brain a new channel for the reception of information from the outside.

The method developed for activating the CIP includes two consecutive stages:
I. The removal of the psychological barrier on the level of the human conscious.
II. The removal of the psychological barrier on the subconscious level.

### I. The Removal of the Psychological Barrier on the Level of the Human Conscious.

### Goal:

The removal of the mystical aspect from “ESP” and the provision of a conscious approach by subjects in mastering information perception from the outside world.
1. First, a demonstration is conducted to show the possibility of information perception. New subjects are given the opportunity to look at special blindfolds which are impermeable to light and do not allow for peeking. Further, these blindfolds are put on the eyes of people who have an activated CIP and a demonstration is conduct of their “unbelievable” capabilities.
   the listing of colors from a thick folder of colored paper
   the reading of any text
   the description of drawings
   the playing of any table games (“tic-tac-toe,” etc)
   walking around the room in between pieces of furniture
   walking around the room one after another, following a volunteer
   games involving balls, boxing
2. After the demonstration subjects are given an introductory lecture, giving the material treatment of the paranormal phenomenon of “ESP”
3. Subjects are familiarized with the “simplified hypothesis” regarding the Information universe and the presence in the human brain which is capable of receiving information directly from the outside world, bypassing the sensory organs.
4. Subjects are familiarized with those possibilities, which open before a person with the mastering of Information perception of the surrounding world.
5. This is followed by responses to questions which may arise.
6. Over the course of further education a series of lectures is conducted on the theory of Information-Energy basis of the material world.

### II. The Removal of a Psychological Barrier on the Subconscious Level.

### Goal:

To overcome the brain's genetic memory of the impossibility of receiving information from the external world except with the aid of the five sensory organs.

### Basic Approach:

The subject is placed under conditions which make it impossible for the brain to receive requested information through traditional pathways. (For this purpose, special approaches and materials are included in the patentable method of activation of Center of Information Perception of the outside world.) The brain is forced to “find” a new channel to collect information. This channel becomes the CIP. The brain starts to receive information from the outside using a new channel under controlled reception conditions.

A positive result, repeated many times, will strengthen the subconscious the reality of information perception, which will ultimately bring the removal of psychological barriers and the activation of the CIP.

### Approaches to be Used:

### 1. “Internal Radar”

The most important and most difficult thing in the early period of CIP activation is to exclude seeing as a pathway for the brain to receive visual information from outside.

The concept of “seeing” is closely associated with the concept of seeing with eyes by our brain. Therefore, when you offer a subject the opportunity to see something, his brain sends a signal to the eyes: “Eyes, what you see?”

The eyes “answer:” “Nothing. We are closed.” The brain “insists” on receiving visual information, since after the demonstration, [the brain] has already, on a conscious level “believed” in this possibility. But the eyes cannot give this information—they are closed. Following the insistence of the brain, the subject tries to see something through closed eyes. He starts to furrow his brows, his eyelids shake. All these attempts are unsuccessful—the eyes cannot see through non translucent physical barriers.

To remove this barrier one must use the “internal radar” method.

The subject is asked to imagine that on his forehead region there is something akin to airport radar, which allows him to use an invisible ray to “sense” and identify objects entering his “field of vision.” Therefore, looking at various objects, it is necessary to look at them not through closed eyed, but with the forehead, that is, with the “internal radar.”

This approach allows one to eliminate attempts to receive visual information with the eyes, and forces the brain to look for other, unorthodox pathways. Activation of the CIP begins.

### 2. “Request-Response.”

The subject is told that looking at an object with closed eyed he must not wait for the brain to receive a visual image of the object. Meanwhile, the brain is not yet familiar with such a pathway for receiving information and is distrustful of it. Only when the brain, at the subconscious level, is convinced of the objectivity of the information it is receiving, does rewire itself (“plasticity”) and try to convert it into visual images.

It is appropriate to explain to the subject that the brain is capable to convert one type of informational concept into another. For example, if you say “Tea Rose,” this auditory information will evoke in the brain a visual image of the flower. This is how information will reach the brain from the CIP in the future.

Until this has happened, the subject is forced to rely on “guessing.” This is aided by the “Request-Response” approach. Naturally, the subject cannot see the object in front of him with closed eyes. He must not try to look at it with closed eyes. He must try to catch the object with his “radar” and ask himself “what is this?” (request). Something will certainly come to mind (response). This must be sounded out.

The brain, aroused by the “question” looks for an opportunity to receive information. Since the only way for it to “pass” in to the brain lies in the CIP, the brain starts to use it, although shyly and uncertainly at first. Since the brain “does not understand” the source of information (non-visual, non-auditory, non-tactile) it groups it with the category of “guesses.”

As the “response-guesses” of the subject correspond with reality more frequently, the brain will “understand” that this source of information can be just as easily trusted, as other sensory organs, and will start to actively use the CIP.

### 3. Forbidding the Activity of ‘Thinking.’”

The reception of information of the surrounding word lies in the realm of feeling. Our consciousness does not interfere with the work of sensory organs.

It merely processes information signals that it is receiving. This guarantees the objectivity (and not the subjectivity) of information arriving from outside and an approach is developed for the million-year long evolution of life on earth.

This “non-interference” of consciousness into CIP activity has not yet been achieved. The brain does not treat this center as a sensory organ and does not unquestioningly trust information that comes through it. It starts to “think” about how real the information reaching it could be, attempting on the level of “consciousness” to find possible alternatives. As it goes through them, it completely loses the correct information it had first received.

It is necessary to “close off” the possibility of such interference by the conscious in the work of the CIP. This is achieved by not allowing the subject to “think” about the question, but is given the opportunity to sound out the information that the brain receives. Only after having sounded it out and having checked how it corresponds to reality, the brain may be convinced that it is working properly, and if not, that it will enter the necessary corrections.

### How to Stop “Thinking”

A human life is composed of constant decision-making. Our brain makes these decisions on the basis of information it receives from the outside. This information is transmitted to the brain by sensory organs: vision, hearing, sense of smell, sense of taste, and sense of touch. The brain receives, as well as processes the incoming information, while consciousness should not, by any means, interfere with the work of sensory organs. This guarantees that the arriving information will be accurate, objective, and the decision which the brain makes will be a correct one. The interference of consciousness with the work of sensory organs is categorically contraindicative. It can transmute into catastrophic consequences.

Scientists-psychologists have conducted the following experiment. Ten people lined up along a wall. Nine of them were in on a collusion, while the tenth was unaware of this. A scientist—a participant of the study—came before the group with a black sheet of paper. He handed this paper to one of the ten people (an accomplice in the collusion) and asked: “What color is the paper?” The participant, without giving the matter much thought, quickly answered: “White”. The second participant of the experiment gave the same answer, as did the third, the fourth . . . When the scientist had reached the tenth person, who was not in on the collusion, and, handing him the black sheet of paper, asked “What color is it?”, the participant assuredly replied: “White!” . . .

When the tenth participant of the study saw that the first person in line referred to a black sheet of paper as white, he was certain this person had made a mistake. This certainty shuddered when the second, the third, the fourth participant had called the sheet of paper white, and when the rest confirmed that the paper is white, he became convinced that it is not them who got it wrong, but him. Although the accurate information that the sheet of paper in front of him is black was being conveyed by the sensory organ—vision, his consciousness had nevertheless interfered with its function, imposing erroneous information.

In the given illustration, false information was artificially imposed from the outside. This happens seldom in real life. Our brain is used to trusting its sensory organs. Behind this “trust” stands the million-year long evolution of life on earth. The brain understands and is accustomed to receiving information by means of five sensory organs.

In the meantime, the pathway of receiving information through the Center of Information Processing (CIP) is incomprehensible for the brain. The information received in this way is recognized by the brain not as something real, but as a product of its own conscious activity, in other words, a fabrication. The brain is unable to draw a line between objective information that is received, and something that it produces itself. It has not yet worked out a mechanism of automatic non-interference into the function of this new sensory organ. Therefore, a formation of such mechanism is needed, a construction of trust by the brain to this new channel of information processing. It is necessary to force the brain not to control the process of receiving information, but to accept this information, as it does, using the habitual sensory organs.

The information received by our brain through the five sensory organs is not susceptible to its thorough examination. If we see a red traffic signal on the road in front of us, we do not stop and think: “ . . . perhaps the signal is green, yellow, or blue?”—we just step on the breaks. Yet if we start to analyze the light and look at it intently, then soon enough, the light may start to seem orange, brown, or even black. If we start thinking about the acquired information, it means that something about it is not quite reassuring for us, we doubt its validity. And then the brain, aiming to please us, begins to feed us various alternatives that would be more suitable. It is no longer concerned about the objectivity of received information, but only about gratifying us. And that is unacceptable.

Generally, the first information signal received by the brain, is always correct. If the light in front of you is red, it will not seem blue first and then red. No, right away, you will see it as red. The same goes for smells, sounds, and other informational signals.

It is also identical to what happens with information incoming through the CIP. The first feeling is always the correct one. And it is crucial to grab hold of it and stop there. During sessions, it is insisted upon that subjects name anything that first pops into their mind without thinking. Any hesitation to answer carries a possibility of starting to doubt.

For instance, I tell one of my subjects to close his eyes; I place a yellow sheet of paper in front of him, asking which color it is. I can confidently say that the first thought that comes to his head will be “Yellow”. And it would be accurate. However, if the subject does not give the answer immediately, then a thought develops in his consciousness: “Maybe it's red? Or blue? Green? Pink? Orange? Blue”. And the longer he will be silent, the more alternative answers will appear in his head. He will lose the objective information, will not be able to recall which thought came to him first, and will relegate to mere guessing.

Therefore, it is insisted upon that subjects answer immediately. The subjects must not “think”. Depending on the way the subject is reassured each time that the answer given without delay reflects reality, his brain gradually fills with trust for this unaccustomed channel of acquiring information from the outside, and the subject stops reexamining it and throwing in new (alternative) answers, and instead begins to actively use this new sensory organ.

### Applicable Materials

Materials applicable to education must be simple, informative, and easily to follow for home exercises. “Informativeness” is to be understood as the volume of information carried.

1) Colored paper—This tool is extremely easy and informative. Its simplicity lies is in the fact that it is a flat, homogenous, sufficiently large in area, monochrome object, which provides its heightened informativeness. In other words, such a sheet of paper carries in its large area simple information—color. This is the ideal material in the early stage of CIP activation.

### 2) Non-translucent plastic cups.

### 3) Newspapers with large headings.

### 4) Children's book with large font and bright pictures.

### 5) A small rubber ball.

### 6) Blindfold

The purpose of the blindfold is to make it impossible to use eyesight to receive visual information.

For exercises and demonstrations a special constructed blindfold is being used. It consists of two thick foam circles around 5 cm in diameter and 1.5 cm thick. Lined with black knitted material (so as not to irritate the eyes) the cups are placed on a long knitted rubber band at a distance from each other equal to the length of the bridge of the nose. This is done so that the bridge of the nose does not elevate the blindfold on the face. In this way the foam circles are brought in close contact with the eye sockets and all penetration of light to the eyes is eliminated.

Exercises aimed at activating the CIP take place under different modes of information perception.

### Subject-Developable Modes of Information Perception

1. “Real sight”—the reception, by the brain, of visual information regarding physical objects in the field of real sight.
2. “Seeing through physical barriers”—The reception, by the brain, of information regarding an object, hidden by a physical barrier.
3. “Farsightedness”—The reception of information, by the brain, of an object located at a significant distance.
4. “Seeing the past”—The reception of information, by the brain, of events that took place in the past.
5. “Retrieving information from any type of carrier.”—The reception of information, by the brain, from such carriers as computer disks, compact disks, tapes, etc.

### A. “Real Sight” Mode

### Mode Basics

The subject must learn how to receive visual information without using his organs of sight, that is, to “see” with closed eyes. Therefore while developing this mode all the exercises are carried out with closed eyes.

### Practice Method for Activating the CIP in the Mode of Real Sight.

### 1. Introductory Individual Exercise.

This method presupposes working with only two colors. The purpose is for the subject to develop his first skill for “turning off” his eyes while looking at sheets of colored paper. Red and yellow colors are recommended.

The subject closes his eyes and is a given a red sheet of paper. Having announced what color it was, the subject is asked to use his “radar” to observe it and to remember the sensations which it evokes. After that the sheet is taken away. The subject keeps his eyes closed until the completion of the exercise.

The same is done with a yellow sheet of paper. This exercise is repeated several times with the red and yellow colors alternated. The subject is always told the color that he is looking at.

After that, a red sheet is put into one hand of the subject and a yellow sheet in the other. He is told which one is where and is asked to compare them.

In the following exercise the subject receives both sheets and is asked to separate them, “observe” them and to name which one of these is red, which one is yellow. This is done several times.

The subject is then given a red and a yellow sheet of paper without being told which one it is and is asked to name them correctly. The colors given are alternated. After each time, the subject is told whether he had correctly “seen” the color he was observing.

Having conducted this exercise 15-20 times, irrespective of the results, the subject is told: “Now I will give you a new color—not yellow or red. Look at it and tell me which color it is.”

It is recommended that Green or Blue be given. Usually the answer is correct. The subject is asked to open his eyes so as to confirm for himself that his answer was correct. This creates a strong impression on the subject and facilitates further education.

The key think is that the subject, in trying to look at the Red and Yellow colors, has already started using his CIP. But while the subject is limited to two colors and knows which two they are he involuntarily “turns on” the conscious and tries to guess which color it is in front of him, without “closely listening” to the signal of his CIP. As soon as the subject, unexpectedly for him, is stripped of the limiting framework and finds himself one on one with the unknown color, he has nothing to choose from. He must “listen closely” to the information signal and name the color. Ninety percent of subjects name the color correctly. Of the remaining 10% of subjects (the ones who made a mistake on their first try) more than half get the color right on their second try. If the subject is not able to get the right color even on the second try, the subject's attention should not be focused on the mistake.

### 2. Group Exercise.

Purpose—methodical prompting of the brain to start using the CIP to receive visual information.

### Working with Colored Paper.

Subjects position themselves around a table. They are asked to close their eyes. A colored sheet is placed on the table. The subjects are asked to look at it, and to raise their hand when they are ready to answer. During the exercise they must continue to sit with their eyes closed.

So that the opinion of one subject cannot affect that of others, the survey must not start until all of the hands are up. After they survey has been complete, the subjects are allowed to open their eyes and see the color of the sheet lying on the desk.

In developing this exercise put 2, 3, and more sheets of colored paper on the desk, asking subjects to not only name the colors but also to identify the position of the sheets on the desk.

### Working with Text.

The subject is asked to close his eyes. He is given a large newspaper headline. The index finger of the subject is placed on the first letter of the first word of the headline. The subject is told the letter and the subject traces it with his index finger. The subject is asked to imagine this letter, to “see” it. Then, his index finger moves under the second letter, and the subject is asked to name it himself. The moderator only says if the letter has been named correctly or incorrectly. The whole word and the entire headline must be read in this way.

### Development of Spatial Sight.

The subject is asked to close his eyes. A plastic cup is placed upside down on the table. The subject is asked “see” it and to place his hand on it. The cup is to be taken away and another put in its place. The position of the cup on the table changes.

In developing this exercise:

### 1) Two cups are placed on the table and the subject needs to see them simultaneously and to place his hands on both of them.

### 2) Several cups are placed on the table and the subject must put them together.

### 3) Small coins are dropped on the table and the subject must pick them up and put them in a cup.

### Developing Information Perception of Moving Objects.

The method takes place in a sufficiently spacious, empty room. The subject is asked to get up and close his eyes. The moderator stands a small distance in front of the subject, with his back to him. The subject must “see” the person standing in front of him. When this happens, the moderator starts to move around the room. The subject follows the moderators, exactly following his route.

In developing this exercise:

1) chairs are placed around the room to increase the difficulty of maneuvering
2) games with balls
3) boxing

### B. “Seeing Through Physical Barriers.”

By way of a prop, a specially prepared stationery envelope made of thick paper is used. A large “window” is cut out on one side of the envelope; the other side is left untouched.

The subjects sit around a table. They are asked to turn away. The moderator puts a sheet of colored paper into the envelope and puts it on the table “window” down. The color of the paper in the envelope cannot be seen.

The subjects are asked to turn to the table to look at the envelope with eyes open. This approach is designed so that the brain of the subject, having confirmed that it is impossible to receive information via this pathway, will no longer “ask” the eyes which color the paper in the envelope is, but will instead concentrate on the reception of signals from the CIP.

Now the subjects are asked to close their eyes and to see with their “inner radar” or in the “request-response” mode the color of the paper in the envelope. They must be told that the non-translucent paper which makes up the envelope is a barrier only for the eyes, but not for the CIP. The eyes close only so that the brain is not distracted from the visual information it is receiving.

Those who are ready to answer open their eyes, but do not name the color, so as not to force it on the others. When all the subjects are ready to answer, the moderator starts the survey. He then turns the envelope “window” up. The subjects can see the color of the paper located there and compare it to their response.

In further developing this exercise:

### 1) Two colors are placed in the envelope and the subject is asked not only to name them, but also to determine which one is on top and which one on bottom.

### 2) A container larger than an envelope is used (a cardboard box, a briefcase, etc.) in which one can put different object for identification.

### 3) The subjects are asked to “look” through the wall and name the color of the paper lying on the table in the next room.

### 4) The subjects are asked under which of the several cups the rubber ball is located.

### C. “Farsightedness”

The subjects are located on one side of the room, the moderator on another. In the upper left hand corner of a clean sheet of paper the moderated writes a number from 0 to 9 without showing it. The subject is asked to close his eyes and to “see” the number with his “internal radar” or with the mode of “request-response.”

The moderator starts the survey when everyone is ready. He gets at least one correct response. He shows the number to the subjects then turns the sheet away and writes a second number. The procedure is repeated.

A new type of memory is developed during this exercise. A “photograph” of the moderator's sheet appears in the subjects' brain with new numbers. This gives the subject the opportunity to “read” the number he is receiving from any number and in any direction. It is important to make sure that this formation takes place in the subject's brain. For this purpose it is necessary, from time to time, to ask the subject to conduct the “reading” of the number from the assigned digit and in the assigned direction. While one subject reads, and the other control and correct him or her.

The number can be brought up to 20-30 variations.

In the development of this exercise:

1) the reading of newspaper headlines from a large distance (over the phone)
2) the reading of words and sentences over the phone from a computer monitor

### D. “Seeing the Past”

The subjects sit around one table. They are asked to turn away. The moderator places a sheet of colored paper on the table, allowing it to lie there, and then taking it away and hiding it.

The subjects are asked to turn towards the table and asked to look at it, so that the brain “knows” that the table is empty and it is not necessary to ask the eyes anything

Now the subjects are asked to close their eyes, to “go into the past” and to see with their “internal radar” or in the mode of “Request-response” the color of the paper lying on the desk. When the answer is ready, the eyes open.

Having asked everyone, the moderator shows the paper lying on the table.

In developing this exercise:
1) two or more sheets of colored paper are placed on the table and subjects are asked not only to name their color, but also to indicate their location on the table.
2) any object can be placed on the table;
3) the moderator pointedly hides some object in the room and the subjects, using the mode of seeing the past, find it.

### E. “Removing Information from any Type of Carrier”

This is one of the more difficult modes of information perception. It presumes a high level of development of the subject's CIP.

At first a demonstration is conducted showing the possibility of reading information on computer disks.

After conducting the demonstration the moderator gives the subject a computer disk which contains some text and asks the subject to read the text at home without putting the disk in the computer. The moderator makes no other indications, only the recommendation to continuously prompt the brain to receive the information on the disk. This is a difficult task in the “request-response” mode which ultimately comes to success.

Only the moderator may verify whether the text was read correctly. If the subject tries to verify the correctness himself and places the disk in the computer, finding there a different text, the assignment will have been ruined, since the text will no longer be unfamiliar to the subject.

In developing this exercise:
1) It is possible to place a photograph on the disk;
2) It is possible to try to work with any other information carrier: an audio tape, a compact disk, a video disk, etc.

### Attributes of the Method for Activation of the Center of Information Processing (CIP) in Blind Persons

The activation method of the CIP in people who are blind differentiates from work with those people, who do not have vision problems. A distinction needs to be made between two different groups of blind subjects.

1^{st} group: seeing people who have lost their vision as a result of an illness or an accident.
2^{nd} group: people who are blind from birth (congenitally blind).

The difference stems from the fact that the brain memory of people from the first group contains visual images of different objects and color spectrum acquired during the period when they still had visual reception and remembered such images. This allows the brain to recognize and connect a visual image or color of an object with the image data of the same object, arriving via the channel of information reception.

The people in the second group (blind from birth) do not contain such images in the brain, and hence are not able to recognize and name that, which they had never perceived in their lives.

With subjects in the first group, a method of operation in the Mode of “Real Sight” is recommended, same as with those subjects that do not have vision problems. The only difference lies in that the sessions (particularly in the first stage—before information reception occurs) need to be conducted not in a group setting, but, rather, individually.

It is apparent that when you work with one member of such a group, the other subjects cannot follow along with what takes place. They become distracted, lose interest in the lesson, and begin to engage themselves in other activities. Only after a successful activation of the CIP in each subject, it becomes possible to unite them in small groups as a way to induce an element of healthy competition, and the effect of contest within the group.

For subjects in the second group, the methodology of the lesson ought to be entirely different. Given that the brain of subjects in this group does not contain visual images of the objects in his surrounding world, it needs to accumulate and memorize them employing not the familiarized channel of vision, but the activated CIP.

For instance, work with a subject can start with a proposal to memorize a sensation of only two colors: Black and White. Intermittently, the subject is given sheets of periodically—black, periodically—white paper, inviting him or her to remember the sensation evoked by each color. Afterward, a set of papers merged into a pack is given, with a task to divide them according to color. The achieved result should be one hundred percent.

As this result begins to occur on a steady basis, a third color (for example, red) can be introduced and again worked on in the same manner. Thus, gradually, the subject's brain will become familiarized with a wide spectrum of colors.

After this, the subject's brain may go on to acquiring images of plane geometric figures: triangles, circles, squares, trapezoids, ovals, star, etc.

Upon a successful completion of this cycle, three-dimensional objects may be introduced: spheres, pyramids, cones, etc.

The accomplishment of the preceding task is followed by the introduction of the blind subjects with the everyday routine surrounding them. The objects of study are chosen by the moderator who guides the subjects.

All sessions with subjects in the second group are conducted strictly on an individual basis.

### Specifics of Activation Method for the Center of Information Processing in the Blind

In the activation method for the Center of Information Processing (CIP), the most concentration focuses on “closing off” the brain's accustomed channel of receiving a given type of information. By means of compelling a subject to receive visual information without using organs of sight, we are leaving his or her brain with a single option—to utilize the channel of direct information reception. This is the key element in the activation method of CIP.

In working with the blind, an equivalent approach is needed: “closing off” of an accustomed channel of information reception. Although in this case, the accustomed channel is not vision, but a sense of touch. Therefore, in working with blind subjects, it is necessary to try and abolish their attempts to touch any learning materials with their hands.

When talking about the blind and ways of teaching them informational processing of the surrounding world, a clear distinction needs to be made between two different groups of these individuals:

First group: seeing people who lost their vision as a result of an illness or an accident.
Second group: people who are blind from birth.

When working with subjects who had the ability to see but lost their vision as a result of an illness or an accident (first group), it is essential to appeal to their memory, summoning them to recall an appearance of a certain color or a certain shape of an object. Learning materials used in the beginning stages of training should be limited to paper of different colors, because even touching them with hands will not give the subject a sense of their color. This awareness allows him or her to cease seeking an answer through senses of touch, and rely only on the information incoming through the CIP.

In working with subjects of the first group, the initial stage of instruction is the most significant. As the brain begins to identify information received through the CIP, we can regard the main goal of teaching accomplished. Further work consists of broadening of the brain's base of visual images, practicing the utilization of this new informational channel in real life, gradual stepping away from orienting oneself in the surrounding world only using the sense of touch, recognition of the notion that one is no longer disabled, and involvement of subjects in social life.

Instructing people in the second group (blind from birth) requires much more exertion, including a different methodological approach. Their memory does not contain any visual images because throughout their life, they were never able to see. Hence, any attempt to make them visually identify information received by the brain is bound to fail. Without a base of visual images in the brain, a person is not able to recognize what is in front of him/her. A moderator is faced with a principally new task: to create a base of visually-informational images of the surrounding world in the subject's brain. In other words, not the visual images which enter our brain through the organs of sight and get memorized, but the visually-informational kind that will enter the brain through a new channel—the CIP. It is possible that the location of these images in the brain will differ from the location of visual images received with the help of eyesight. An answer to this supposition should be found as a result of the following scientific study.

As in the case of working with subjects in the first group, the lesson needs to begin with the examination of two colors. Yet, unlike with subjects who lost their sight, the only colors recommended here are Black and White. This suggestion is based on the notion that people who never had the ability to see will later need to grasp such notions as Light-Dark, and it is best that their visually-informational processing begins precisely with the black and white colors.

A moderator places a black sheet of paper on the table in front of the subject and, naming it, invites the subject to remember the sensation evoked by this color. One needs to be ready that the subject will likely say: “But I do not feel anything.” And he need not feel anything.

We “feel” only when the brain receives information through its customary channels. We are familiar with a sense of light, when visual information enters through the organs of sight. Our sensation of hearing, smell or taste signals will not be mistaken for anything else. And this is absolutely necessary, because these signals penetrate our brain so that it would react to them.

A signal entering through the CIP is entirely new and unfamiliar to our brain. It is indispensable to learn how to identify it. This signal reveals itself in a form of a thought, suddenly appearing in one's head. To differentiate this signal, to set it apart from all of the other thoughts, which spontaneously and continually rush through our head, is extremely difficult. Yet it is precisely this, which comprises the meaning of teaching subjects to use a new channel of receiving information. They need to assuredly distinguish objective informational signal-thought from their random thoughts.

Going back to instructing a subject who is blind from birth, do not be surprised at his answer: “But I do not feel anything.” Explain to him that it is not required that he feel anything specific. He simply needs to know (as per your words) that what lies in front of him is a black sheet of paper. Simply know. And now, in front of him is a white sheet of paper. Let it remain there. Allow the subject the time to adapt to its information.

Do not, by any means, permit the subject to lean down toward the paper laying on the table, nor, if the paper is in his or her hands—to bring it toward his or her face. It is the information grounded in the genes, that the attainment of a visual image can only be done using eyesight, which compels the subject to bring the object of study closer to the eyes. This is even relevant to those, who are born blind and have never used organs of sight in their lives. Such an attempt needs to be halted undeniably and immediately.

In alternating black and white sheets of paper, the moderator facilitates a sense of difference between these two colors. When, in his opinion, a recognizable understanding about the distinction of these colors has been formed in the subject, the second stage of the initial lesson can begin: placing a sheet of paper in front of the subject without naming its color, and asking what color it is. It is important to monitor that the subject names the very first thought that comes to his/her mind. Do not allow the subject to contemplate various alternatives of the answer, because the answer given after such pondering will be the result of conscious activity of the brain, and not the result of objective informational signal received from the outside.

It is also not advisable to establish any sort of time-restraint on developing a sense of distinction between the white and black colors. This is the most imperative aspect of CIP activation. After learning to accurately differentiate between these two colors, it will be much easier for the brain to form visually-informational images of other colors, geometric forms, and various objects. The establishment of the first visually-informational image in the brain of an individual, who is blind from birth, opens the channel for the development of all further images.

Once this fundamental stage is completed, the work on broadening of visually-informational image base in the brain of the blind individual can begin, as well as the further work on his or her adaptation to the life in the world surrounding us.

### EXPERIMENTAL RESULTS

### Example 1

A Neurometric evaluation was conducted on a male subject including a twenty minute recording of eyes closed, with resting EEG recorded from standardized electrode positions over 19 brain regions while the subject sat blindfolded, initially at rest (condition A) and then while trying to read while his eyes were covered with a blindfold (condition B). After visual inspection of each EEG record, two minutes of artifact-free data were selected from A and from B for quantitative (QEEG) analysis. In addition, in both conditions A and B, visual evoked potentials (VEPs) were elicited by binocular visual pattern reversal stimuli.

### QEEG Findings:

The EEG in condition A was of relatively low amplitude. The QEEG shows the frequency distribution expected from a normal child of the subject's age, when recorded at rest with eyes closed and essentially all quantitative values were well within normal limits. There was an expected peak at 10.53 Hz in the middle of the so-called “alpha” frequency band, with maximum values as expected in the visual cortical regions. The generators of this frequency were shown by three-dimensional source localization to be in the bilateral occipital and temporal gyri and the bilateral inferior frontal cortex.

The EEG in condition B showed a completely different frequency distribution characterized by very large electromyogram activity in all regions of the prefrontal cortex and the anterior temporal lobes. The QEEG revealed a great anterior excess of beta activity in those regions. There were large peaks of beta activity (133-18 Hz) in the periorbital, dorsolateral and mesial prefrontal cortex, consistent with wrinkling the forehead and squinting of the eyes. There were no signs of activation in the visual associations regions (posterior temporal and parietal cortex) or in the primary visual (occipital) cortex. The generators of these beta frequencies were shown by three-dimensional source localization to be in the bilateral prefrontal and temporal regions associated with jaw clenching and forehead-eyelid muscle contraction, but there was no sign of activation of the readings required to mediate reading or visual scene analysis.

Strong, normal VEPs were elicited by the pattern reversing visual field in the eyes open condition A, with morphology and topographic distribution as expected from a normal child when tested with open eyes. Inexplicably, a strong and well replicated VEP was detected in the occipital regions in two replicated trials with the eyes blindfolded, in condition B.

### Interpretation:

The conventional EEG, QEEG and brain imaging all indicate strong activation of the frontalis, obicularis and temporalis muscles during the periods when the child was attempting to read. There was no indication that the brain regions concerned with processing visual information were activated during these endeavors in condition B. Perfectly clear, morphologically and topographically normal visual evoked potentials were elicited in with eyes open as well as blind-folded. There was no brain imaging evidence that indicated any input to the visual regions while blindfolded.

### Example 2

A Neurometric evaluation was conducted on a female subject including a twenty minute recording of eyes closed, resting EEG recorded from standardized electrode positions over 19 brain regions while the subject sat blindfolded, initially at rest (condition A) and then while trying to identify colors and to read while her eyes were covered with a blindfold (condition B). After visual inspection of each EEG record, two minutes of artifact-free data were selected from A and from B for quantitative (QEEG) analysis. In addition, in both conditions A and B, visual evoked potentials (VEPs) were elicited by binocular visual pattern reversal stimuli.

### QEEG Findings:

The QEEG was basically similar whether the child was at rest (A) or trying (unsuccessfully) to identify colors and to read (B). The QEEG was characterized by a slight deficit of delta and theta activity (not statistically significant) and beta activity in the normal range, dominated by strong alpha activity which increased in some regions with the effort of reading. In the three dimensional brain source localization images, essentially identical brain regions were activated in the two conditions, although a slightly increased power in the peak at 10.53 Hz of the very narrow band over regions on the left hemisphere was observed in condition B. Asymmetry in the occipital regions in both A and B is reflected in numerical values, especially in slow delta waves, and the two visual regions on the two hemispheres became somewhat desynchronized in B. These changes may be correlates of eye movements, but little or no change occurred in the parieto-temporal regions of the visual association areas known to be engaged in reading.

The technician noted that when the electrodes on her head were disconnected from the recording apparatus and she was told that the recording was finished, the child stood up and walked unaided and unhesitatingly out of the raised recording booth and stepped down to the floor, although she was still blindfolded.

Only weak, unreproducible VEPs and much phase-locked alpha ringing was elicited by the pattern reversing visual field in the eyes open condition A, as if the child were inattentive without a fixed gaze when tested with open eyes. Inexplicably, a strong and well replicated VEP was detected in the occipital regions in two replicated trials with the eyes blindfolded, in condition B.

### Visual Examination of the EEG Tracings:

The EEG sample collected for the QEEG evaluation was not optimally suited for recognition of possible seizure disorders. Nonetheless, the entire EEG sample was examined visually for quality control prior to quantitative analysis. It was very difficult to evaluate condition B, because of the pervasive artifacts generated by constant scanning eye movements. While it was not the purpose of this evaluation, it should be mentioned that the overall profile of this child in both conditions A and B bore resemblance to the QEEG profiles often associated with learning disabilities.

Interpretation: The QEEG data strongly support the absence of visual functioning in this child subject in the blindfold condition, especially the anomalous increase in alpha activity when blinded and trying to “read”, which a mass of published articles agree should be accompanied by EEG desynchronization and a correlated increase in beta activity. The data showed no increase of beta except for the EMG in the periorbital regions in B, due to the forehead musculature, and a clear increase of alpha in the source localization images at 10.53 Hz. On the other hand, there was a mysterious, clear and replicated VEP in condition B, even though poor VEP reactivity was found in condition A with no blindfold and eyes open.

### Example 3

During the experimental process a dynamic control of the subject children's condition was performed by methods of EEG, gaseous discharge visualization (GDV) and behavioral observation.

During these experiments by the inventor, classes were performed to teach Informational Visualization to two groups of blind and visually impaired children aged 8-11 and 16. The lessons were conducted from 3:00 pm to 5:30 pm in a regular room with observers. The subjects selected for the experiment did not have any obvious psychological abnormalities. The classes were conducted after an explanation was given to the subjects and with their understanding and full agreement.

The experiments demonstrated the following major results:
1. All visually impaired children aged 8-11 with whom the study was conducted showed an ability to recognize images (reading text) and orientation in the room without using direct vision (i.e. wearing a blindfold).
2. Blind children, aged 16, showed an ability to recognize different colors of paper shown to them by the inventor in a random order. Differentiation was made between 6 colors.
3. As the Informational Visualization educational process progressed the number of correct answers increased from 20-30% to 90-100% among different subjects.
4. EEG and GDV recordings were conducted both prior to the study and during the study.
5. The results of the GDV conducted during the educational process among different children showed a significant improvement of psychosomatic condition—a transformation from energy deficient condition in one or both children's hands in the initial stage of the experiment to a stable condition after attending 2-3 days of classes.

### CONCLUSIONS

This invention provides obvious advantages and features to aid individuals in improving their vision cognitive abilities and enjoying increased success in their lives. It is a practical system and method to incorporate into daily activities in individual situations, in group situations, and in organizations.

The system and method enables individuals to develop a part of the brain which is typically underutilized with resultant development of new channels to attain vision, while simultaneously potentially increase IQ and improve memory. The invention will allow blind people to regain partial or full vision without any mechanical or technological means and represents a way to develop sensory perception that has been dormant.

While the above descriptions contain many specific details, these should not be construed as limitations on the scope of the invention. There are many possible variations of using, interpreting, and applying this invention. Accordingly, the scope of this invention should be determined not by the embodiment(s) illustrated, but by the appended claims and their legal equivalents.

While the present invention has been described with particular reference to certain embodiments of the system and method of the invention, it is to be understood that it includes all reasonable equivalents thereof.

What is claimed:

1. A method for altering a visual cognitive ability in a human comprising:
(a) eliciting the human to imagine that he or she possesses an optical sensory ability that operates when the human is sight-deprived;
(b) presenting the sight-deprived human with an optical sensory stimulus for a time sufficient that the human perceives the presence of the optical stimulus; and
(c) eliciting, within a few seconds of the human perceiving the presence of the optical stimulus, a description of the optical stimulus,
so as to thereby alter the visual cognitive ability of the human.

2. The method of claim 1, wherein in step (c) initiation of the description is elicited within 5 seconds.

3. The method of claim 1, wherein in step (c) initiation of the description is elicited within 2.5 seconds.

4. The method of claim 1, wherein the optical stimulus is paper which is substantially black.

5. The method of claim 1, wherein the optical stimulus is paper which is substantially white.

6. The method of claim 1, wherein the optical stimulus is paper which is substantially colored.

7. The method of claim 1, wherein the optical stimulus is text.

8. The method of claim 1, further comprising, prior to step (a), the steps of:
(i) exposing the human to a demonstration of a second human describing an optical stimulus, wherein the second human is denied sight of the optical stimulus; and
(ii) applying a sight-depriving means to the human.

9. A method for eliciting a visual cognitive ability in a congenitally blind human comprising:
(a) eliciting the human to imagine that he or she possesses an optical sensory ability;
(b) (i) presenting the human with a first optical stimulus for a time sufficient that the human perceives the presence of the optical stimulus and (ii) informing the human of the nature of the optical stimulus;
(c) eliciting the human to familiarize himself/herself with a thought elicited by the optical stimulus;
(d) repeating steps (b) and (c) with a further optical sensory stimulus;
(e) repeating steps (b) to (d) a sufficient number of times until the human is able to identify the nature of the optical stimulus without being informed of its nature,
thereby eliciting the visual cognitive ability in the congenitally blind human.

10. The method of claim 9, wherein the first or further optically stimulus is paper which is substantially black.

11. The method of claim 9, wherein the first or further optically stimulus is paper which is substantially white.

12. The method of claim 9, wherein the first or further optically stimulus is paper which is substantially colored.
